(19) United States
(12) Patent Application Publication
(10) Pub. No.: US 2023/0111867 A1
(43) Pub. Date: Apr. 13, 2023

(54) Electrically Heated Wearable Belt for Reducing Fat

(71) Applicant: Susan Coats Price, Mt. Olive, NC (US)

(72) Inventor: Susan Coats Price, Mt. Olive, NC (US)

(21) Appl. No.: 17/735,502

(22) Filed: May 3, 2022

Related U.S. Application Data
(60) Provisional application No. 63/253,272, filed on Oct. 7, 2021.

(51) Int. Cl. A41F 9/00; A41D 13/005

(52) U.S. Cl. CPC A41F 9/00; A41D 13/0051

(57) ABSTRACT

The present invention relates to an electrically heated wearable belt and method for applying heat to a part of a user’s body for reducing fat and weight by facilitating generation of sweat. The belt is flexible, foldable, and insulating and includes a heating element in the form of flexible metal strips that receive electrical power from a rechargeable battery. The battery is positioned inside a pocket of the belt and is coupled to the heating element through a connector. The heating element may provide a uniform heating or differential heating to the skin of the user where the belt is worn. The belt has a fastening mechanism for securing the belt and can be applied to the abdomen, hips, thighs, and more of the user.

### CROSS-REFERENCE TO RELATED APPLICATION

The present application claims priority to, and the benefit of, U.S. Provisional Application No. 63/253,272, which was filed on Oct. 7, 2021 and is incorporated herein by reference in its entirety.

### FIELD OF THE INVENTION

The present invention relates generally to wearable belts. More specifically, the present invention relates to a novel electrically heated belt for wearing around a body part and facilitating sweating for trimming fat. The belt has a heating element disposed therein that applies heat to help generate sweat and cut away fat while doing any activity. The belt is insulating and includes a rechargeable battery for providing power to the heating element. Accordingly, the present disclosure makes specific reference thereto. Nonetheless, it is to be appreciated that aspects of the present invention are also equally applicable to other like applications, devices, and methods of manufacture.

### BACKGROUND

By way of background, most individuals live a sedentary lifestyle spending a major portion of their time in doing sedentary activities. Many individuals work in a sitting position using a computer and do other sedentary activities like television viewing, playing video games, and more. Sedentary lifestyles are often attributed to weight gain, accumulation of fat, and even obesity. Individuals who are unable to regularly exercise can also gain weight and fat and are also disappointed with their appearance due to gaining significant amounts of fat. Excess abdominal fat-particularly visceral fat that surrounds organs and puffs stomach into a “beer gut” may act a predictor of heart disease, Type 2 diabetes, insulin resistance, and some cancers.

Many individuals do not like to exercise at gyms and even at home. It is a common observation that individuals are reluctant to do exercises and want an easy method of reducing weight and getting rid of fat. Standard exercises including resistance training, high intensity interval training (HIIT) workouts, core workouts, and more are not effective in reducing fat especially around waist and hips. Exercises may help in losing weight but shedding the stubborn fats in and around the belly and hip regions can be tormenting. Individuals end up spending a lot of money and time without getting desired results. Individuals desire a device and a method of easily losing unwanted weight and fat without exercising and can be used virtually anywhere by a user. Sweating helps in losing weight and fat, but exercises do not help in generating enough sweat to cut fat.

Therefore, there exists a long-felt need in the art for an inexpensive device that can be used by individuals for reducing fat and weight around target body parts such as, the abdomen and the hips. There is also a long-felt need in the art for a fat trimming device that can reduce weight and trim fat without requiring a user to exercise. Additionally, there is a long-felt need in the art for a fat trimming device that can be worn like a belt by users. Moreover, there is a long-felt need in the art for an exercise belt that is easy to use and wear. Further, there is a long-felt need in the art for a fat reducing belt that can apply heat to the skin of a user without any discomfort. Further, there is a long-felt need in the art for a wearable belt that helps to increase weight loss while sedentary and/or while exercising. Finally, there is a long-felt need in the art for a user-friendly and effective sauna belt that generates sweat and helps in facilitating fat loss during any type of activity.

The subject matter disclosed and claimed herein, in one embodiment thereof, comprises an electrically heated wearable fat trimming belt. The belt applies heat to the skin of a user to facilitate sweat and thus helps in reducing fat and weight without requiring a user to do any exercise. The belt further comprising an elongated body made of latex-free material and having neoprene lining for effective heating and insulation, the body having a first end and a second end, the first end and the second end having fastening mechanisms for securing the belt around a body part of a user, a flexible heating element extending from the first end to the second end and disposed inside the body, a rechargeable battery for providing electric power to the heating element wherein the heating element is configured to produce temperatures from about 107° F. to about 135° F. for facilitating sweating of the target body area to reduce fat and weight.

In this manner, the fat trimming sweat facilitating belt of the present invention accomplishes all of the foregoing objectives and provides users with a unique belt capable of helping individuals lose unwanted weight via sweating. The belt generates and maintains heat application to the target body part or area, for example, the abdomen to help sweat fat away while being sedentary or during exercise routines. The belt is easy to wear and is flexible and insulating.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an electrically heated wearable fat trimming belt. The belt further comprising an elongated body made of latex-free material and having neoprene lining, the body having a pair of opposing ends including a first end and a second end, the first end having a loop fastener positioned on the interior surface of the body, the second end having a hook fastener positioned on the exterior surface of the body wherein the loop fastener and the hook fastener facilitate securing of the belt around a target body part of a wearer, a flexible heating element extending from the first end to the second end and disposed inside the body, a rechargeable battery for providing electric power to the heating element wherein the heating element is configured to produce temperature from about 107° F. to about 135° F. for facilitating sweating of the target body area to reduce fat and weight.

In yet another embodiment, the heating element is made of a flexible metal wire.

In yet another embodiment, the heating element is made of a flexible metal strip.

In yet another embodiment, the battery is positioned inside a pocket wherein the pocket is disposed on an exterior surface of the belt.

In yet another embodiment, the battery is a 450 mA Lithium-ion battery.

In yet another embodiment, the battery is releasably connected to the heating element using a connecting wire.

In yet another embodiment, a method for applying heat to a target body part of a user for facilitating sweating to reduce fat and weight is described. The method includes the steps of providing a portable wearable heating belt, the belt including a heating element running along length of the belt, a fastener mechanism for securing the belt around a body part of the user, a rechargeable battery for providing electric power to the heating element, a temperature controller for controlling temperature of the heating element; wearing the belt around the body part; pressing an activation button of the belt to actuate heating of the heating element disposed in the belt; and, applying a uniform heat to the abdomen and stomach of the wearer for facilitating sweating and thereby reducing fat.

In yet another embodiment, a portable sauna belt for reducing fat around an abdomen is disclosed. The belt includes an insulating body made of latex free material having a neoprene lining, a heating element configured to produce less heat in one portion of the body and more heat in a remaining portion of the body, a hook and loop fastener for securing the belt around the abdomen of a user, a temperature controller for controlling temperature of the heating element, a rechargeable battery for providing power to the heating element, and an activation button for transmitting the power from the battery.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the fat trimmer exercise belt of the present invention in accordance with the disclosed architecture;

FIG. 2 illustrates another perspective view of the fat trimmer exercise belt of the present invention in accordance with the disclosed architecture;

FIG. 3 illustrates an exploded view of the fat trimming belt of FIG. 1 in accordance with the disclosed architecture;

FIG. 4 illustrates a perspective view showing a user securing the sauna belt around the waist for reducing fat in accordance with the disclosed architecture;

FIG. 5 illustrates a perspective view of a user wearing the sauna belt of the present invention in an actuated mode for facilitating sweating to reduce fat around the abdomen area in accordance with the disclosed architecture; and

FIG. 6 illustrates a perspective view of another embodiment of the sauna belt of the present invention in an unfolded state in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an inexpensive device that can be used by individuals for reducing fat and weight around target body parts such as the abdomen and the hips. There is also a long-felt need in the art for a fat trimming device that can reduce weight and trim fat without requiring a user to exercise. Additionally, there is a long-felt need in the art for a fat trimming device that can be worn like a belt by users. Moreover, there is a long-felt need in the art for an exercise belt that is easy to use and wear. Further, there is a long-felt need in the art for a fat reducing belt that can apply heat to the skin of a user without any discomfort. Further, there is a long-felt need in the art for a wearable belt that helps to increase weight loss while sedentary and/or while exercising. Finally, there is a long-felt need in the art for a user-friendly and effective sauna belt that generates sweat and helps in facilitating fat loss during any type of activity.

The present invention, in one exemplary embodiment, a novel electrically heated portable sauna belt for reducing fat around the abdomen or any other targeted body part is disclosed. The belt includes an insulating body made of latex free material having a neoprene lining, a continuous heating element configured to produce less heat in one selected portion of the body and more heat in another remaining portion of the body, a hook and loop fastener for securing the belt around the abdomen of a user, a temperature controller for controlling temperature of the heating element, a rechargeable battery for providing power to the heating element, and an activation button for transmitting the power from the battery.

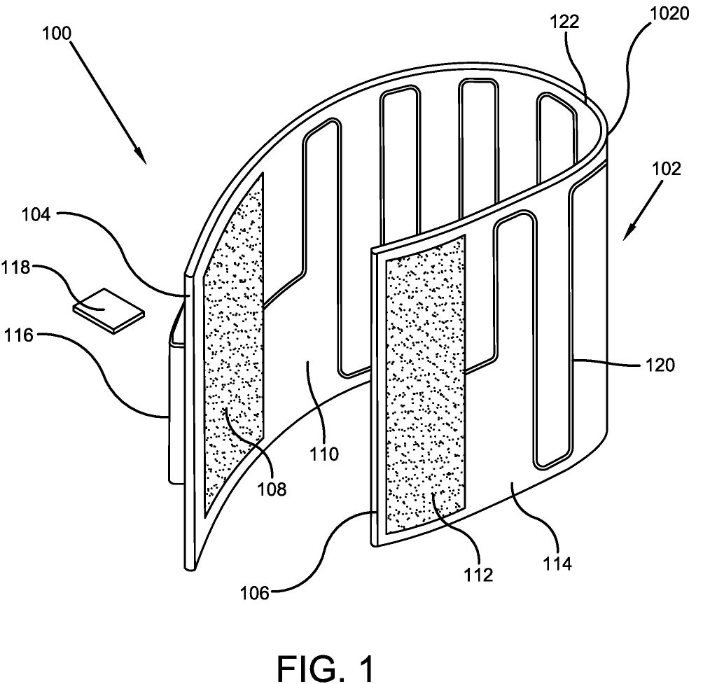

Referring initially to the drawings, FIG. 1 illustrates a perspective view of one potential embodiment of the fat trimmer exercise belt of the present invention in accordance with the disclosed architecture. The fat trimmer exercise belt 100 of the present invention is designed as a unique belt that can be worn around the waist and the hips by a user helping to lose unwanted weight and promoting sweating around targeted areas of the body. The exercise belt 100 generates and maintains heat application to the target body part such as the abdomen to help sweat fat away while sedentary and/or exercising. The belt 100 can be used virtually anywhere and anytime by users by comfortably wearing around the abdomen.

The fat trimmer belt 100 is made of a 100% high quality latex-free compression band with a neoprene inner lining for thermal therapy and soft tissue support. More specifically, the belt 100 has a latex-free body 102 having opposing ends, a first end 104 and an opposing second end 106. The first end 104 has a loop fastener 108 disposed on the interior surface 110 of the body 102 and the second end 106 has a hook fastener 112 on the exterior surface 114 of the body 102 for facilitating attachment of the body 102 around the abdomen of a user. The ends 104,106 can include other attachment mechanisms, such as magnets, buckles, adhesives, and more.

A pocket 116 is sewn on the exterior surface 114 at the first end 104 for accommodating a rechargeable battery 118. The rechargeable battery 118 is removably stored in the pocket 116 and is configured to provide electric power to the heating element 120 disposed in the body 102. The battery 118 can be one of zinc-carbon, zinc chloride, alkaline, zinc-manganese dioxide, nickel, lithium ion, lithium-copper oxide, and more. Preferably, the battery 118 is a 450 mA battery, and once fully charged, can support the heating element 120 for more than 10 days (45 minutes per day). The heating element 120 is in the form of flexible and continuous metal grid that runs along the length of the body 102 from the first end 104 to the second end 106 and is accommodated in a cavity 122 disposed between the exterior surface 114 and the interior surface 110.

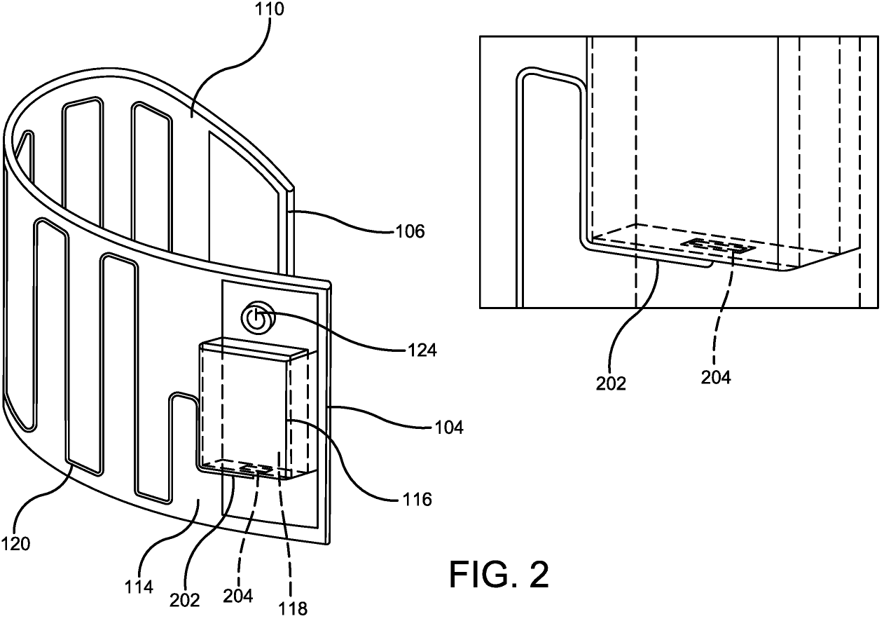

An activation button 124 (shown in FIG. 2) is disposed on the exterior surface 114 preferably above the pocket 116 that is configured to actuate heating of the heating element 120. Electric power is transferred from the battery 118 to the heating element 120 when the activation button 124 is pressed, thereby enabling the heating element 120 to produce heat and thus cause bodily sweating to the wearer of the belt 100.

In the present embodiment, the heating element 120 continuously and uniformly provides heating. The belt 100 can be worn by a user during any activity and thus helps to increase weight loss while sedentary and/or while exercising. The neoprene lining 1020 is from about 2 mm to about 6 mm in thickness and is designed to maximize heating effect of the belt 100. The belt 100 is highly elastic, soft, and comfortable and can be worn easily and selectively inside the clothes or over the clothes by a wearer.

The belt 100 compresses and therapeutically heats the abdominal skin and muscles and therefore trims fat and increases muscle stability and rapid recovery. The latex-free compression band with neoprene lining provides insulation to the wearer and thus is safe for use. One skilled in the art will appreciate that the illustrated fat trimmer belt 100 is merely representative of one type of heating pad and that the disclosure is not limited to the illustrated shapes and/or configurations in the Figures.

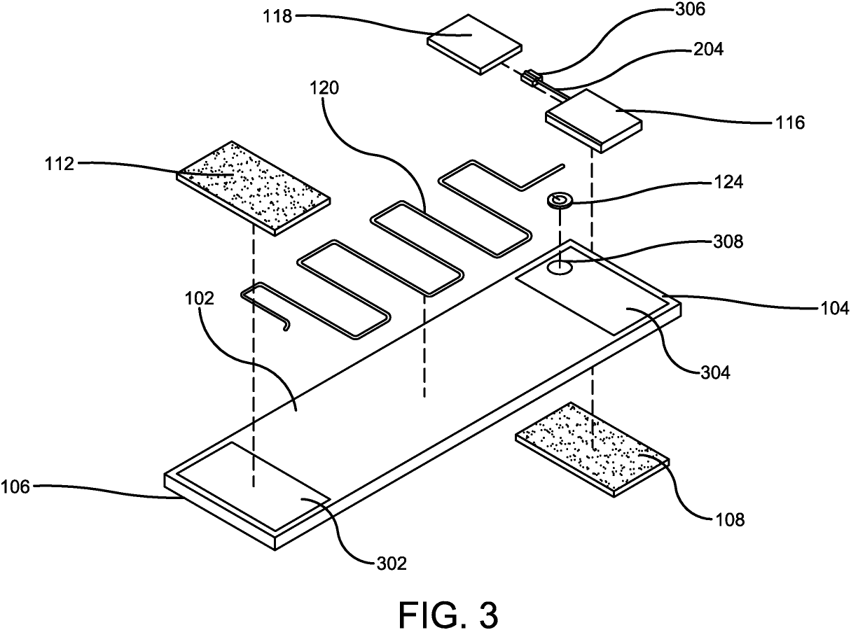

FIG. 2 illustrates another perspective view of the fat trimmer exercise belt 100 of the present invention in accordance with the disclosed architecture. As illustrated, exterior surface 114 has an activation button 124 configured to facilitate transmission of electric power from the battery 118 to the heating element 120 through a connecting wire 202 attached to a power slot 204 of the battery 118. The connecting wire 202 is sewn inside the belt 100 and connects the heating element 120 and the power slot 204 of the battery through a connector (shown in exploded view in FIG. 3).

The pocket 116 is dimensioned to accommodate the rechargeable battery 118 and the battery 118 can be removed from the pocket and the connecting wire 202 and is recharged using a conventional USB cord.

It should be understood that the fat reducing belt 100 comes in a variety of sizes and can be stretched to fit around the waist of a user. The break elongation (i.e., elastic failure) of the belt 100 is in the range of about 400% to 1200% relative to the static length of the belt 100. The break elongation is a percentage of elongation of the belt from a non-elongated or resting state (i.e., static length) before breakage or tearing occurs.

FIG. 3 illustrates an exploded view of the fat trimming belt 100 of FIG. 1 in accordance with the disclosed architecture. The body 102 of the fat reducing belt 100 is planar and flexible and is configured to fold for easy carriage and storage. The loop fastener 108 is adhered on the interior surface at a slot 304 at the first end 104. The activation button 124 is slotted in a circular hole 308 on the exterior surface and is coupled to the connecting wire 202 enabling the transfer of electric charge to the heating element 120.

The hook fastener 112 is adhered and positioned on a slot 302 on the exterior surface such that the hook fastener 112 fastens to the loop fastener 108 for securing the belt 100 around the abdomen of a user. As illustrated, the connecting wire 202 has a connector 306 that can be accessed from the pocket 116 and is inserted into the battery power slot 204 of the battery 118 enabling an electric connection of the heating element 120 with the battery 118 and thereby providing power to the heating element 120 for heating.

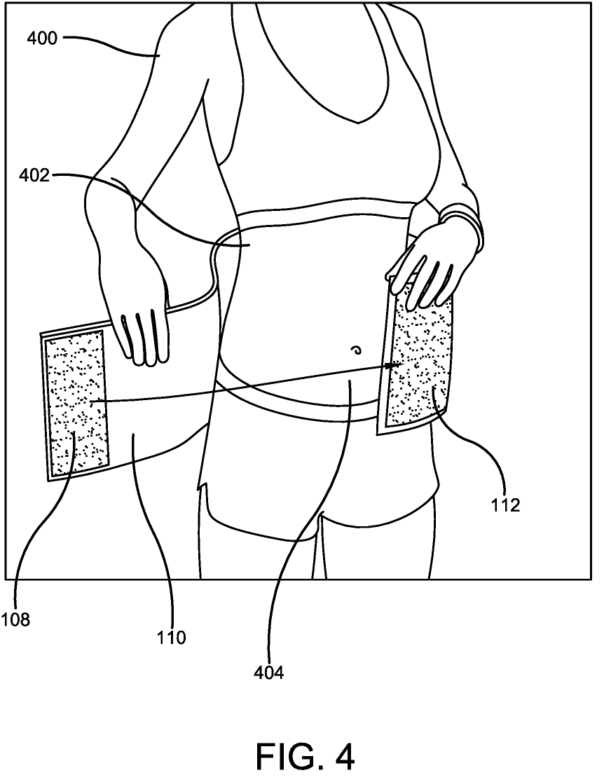

FIG. 4 illustrates a perspective view showing a user securing the sauna belt 100 around the waist for reducing fat in accordance with the disclosed architecture. For securing the belt 100 around abdomen 402, the belt 100 is wrapped around the waist securely and is fastened in front of the stomach 404 by fastening the hook fastener 112 and the loop fastener 108. The belt 100 is flexible and stretchable and thus secures comfortably around waist of the user 400 and enabling a user to do any activity while wearing the belt 100.

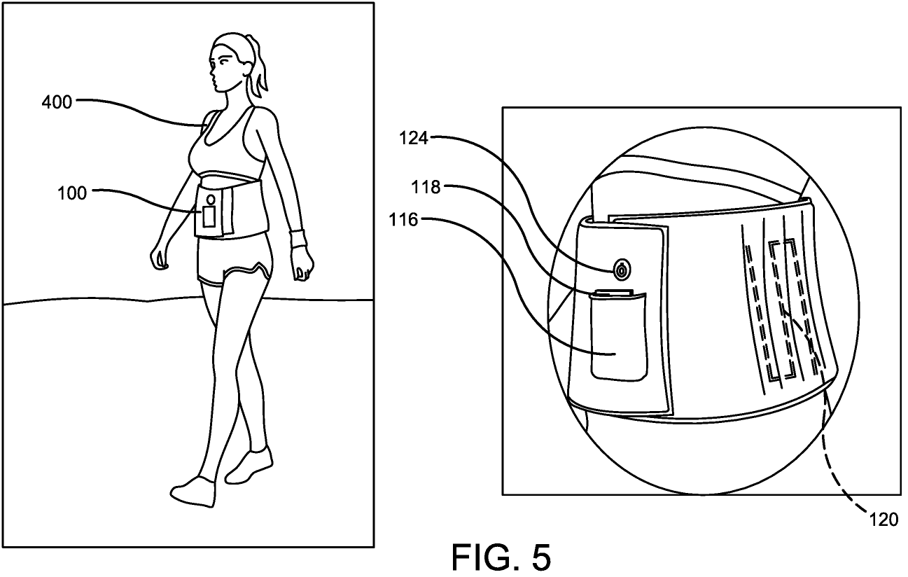

FIG. 5 illustrates a perspective view of a user wearing the sauna belt 100 of the present invention in an actuated mode for facilitating sweating to reduce fat around the abdominal area in accordance with the disclosed architecture. As illustrated, the user 400 wears the electrically heated wearable belt 100 around the abdomen. When the activation button 124 is pressed, electric power is transferred from the battery 118 stored in the pocket 116 to the heating element 120. The abdomen of the user 400, upon actuation of the belt 100, starts sweating thereby reducing or trimming the excessive fat therearound. The heating element 120 can provide a constant temperature of 107° F. and can range up to 135° F. for facilitating sweat. The belt 100 is machine washable and can be available in a variety of colors, designs, and sizes.

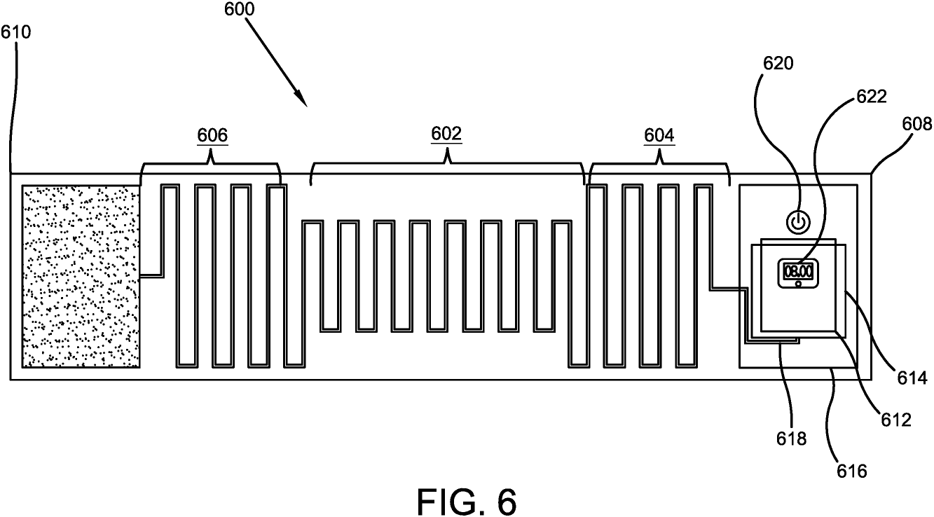

FIG. 6 illustrates a perspective view of another embodiment of the sauna belt of the present invention in an unfolded state in accordance with the disclosed architecture. The sauna belt 600 of the present invention includes a smaller heating element 602 around the center of the belt 600 for providing less heat and includes bigger heating elements 604,606 toward the first end 608 and second end 610 respectively for providing more heat. The belt 600 is designed especially for individuals having stubborn fat on the sides and hips and less fat on the back and/or the stomach. A battery 612 is stored in the pocket 614 positioned on exterior surface 616 and is configured to provide electric power to the heating elements 602,604,606. It should be noted that the heating elements 602,604,606 are continuous and connected to the battery 612 using a connecting wire 618.

The fat reducing belt 600 has an activation button 620 for turning on heating of the heating elements and has a temperature controller 622 for controlling temperature of the heating elements 602,604,606. The temperature controller 622 can be in the form of a knob which can be turned to adjust the temperature. Similar to other embodiments of the present invention, the belt 100 has hook and loop fasteners at opposing ends for fastening the belt around the abdomen.

It should be noted that electrically heated wearable belt 100,600 may come in smaller lengths and can be used for reducing fat on thighs and arms. Further, the belts 100,600 do not use any external power supply and thus can be used virtually anywhere by a wearer.

The invention also discloses a method for applying heat to a target body part of a user for reducing fat. The method includes the steps of wearing a fat trimming belt around the abdomen, pressing an activation button of the belt to actuate heating of heating elements disposed in the belt, and applying a uniform heat to the abdomen and stomach of the wearer for facilitating sweating and thereby reducing fat.

The fat trimming belt 100,600 of various embodiments of the present invention is insulating, facilitates sweating and does not require a user to exercise for reducing fat and weight. The heating elements provide a temperature high enough to facilitate sweating without discomfort to the wearer.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “fat trimming belt”, “sauna belt”, “belt”, “fat reducing belt”, and “electrically heated wearable belt” are interchangeable and refer to the electrically heated fat reducing wearable belt 100 of the present invention.

Notwithstanding the foregoing, the electrically heated fat reducing wearable belt 100 of the present invention can be of any suitable size and configuration as is known in the art without affecting the overall concept of the invention, provided that it accomplishes the above-stated objectives. One of ordinary skill in the art will appreciate that the electrically heated fat reducing wearable belt 100,600 as shown in the FIGS. are for illustrative purposes only, and that many other sizes and shapes of the electrically heated fat reducing wearable belt 100,600 are well within the scope of the present disclosure. Although the dimensions of the electrically heated fat reducing wearable belt 100,600 are important design parameters for user convenience, the electrically heated fat reducing wearable belt 100,600 may be of any size that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A fat trimmer exercise belt for administering a thermal therapy and soft tissue support to a target body area of a user, the fat trimmer exercise belt comprising:
a compression band; and
a heating element for wearing around the target body area;
wherein said compression band is comprised of a neoprene inner lining having a cavity disposed between an exterior surface and an interior surface of said fat trimmer exercise belt for retaining said heating element;
wherein said heating element comprises a flexible and continuous metal grid extending along a length of said compression band from a first end to an opposing second end;
wherein said heating element maintains a temperature from about 107° F. to about 135° F. for generating and maintaining heat application to said target body area for increasing a temperature of said target body area; and
further wherein said target body area includes an abdominal area and a hip of the user.

2. The fat trimmer exercise belt of claim 1, wherein said first end includes a loop fastener disposed on said interior surface of said compression band and said second end includes a hook fastener on said exterior surface of said compression band for attachment of said compression band around the abdominal area and the hip of the user.

3. The fat trimmer exercise belt of claim 2 further comprising a pocket sewn on said exterior surface for accommodating a rechargeable battery, wherein said rechargeable battery is removably stored in said pocket and provides electric power to said heating element.

4. The fat trimmer exercise belt of claim 3, wherein said battery is a 450 mA battery.

5. The fat trimmer exercise belt of claim 4 further comprising an activation button disposed on said exterior surface for actuating said heating element, wherein an electric power is transferred from said battery to said heating element when said activation button is pressed for enabling said heating element to generate a heat to said target body area.

6. The fat trimmer exercise belt of claim 5, wherein said neoprene lining is from about 2 mm to about 6 mm in thickness.

7. The fat trimmer exercise belt of claim 6, wherein said compression band has an elastic failure in the range of about 400% to about 1200% relative to a static length of said compression band.

8. A fat trimmer exercise belt for administering a thermal therapy and soft tissue support to a target body area, the fat trimmer exercise belt comprising:
a compression band; and
a heating element for wearing around the target body area of a user;
wherein said compression band is comprised of a neoprene inner lining having a cavity disposed between an exterior surface and an interior surface of said fat trimmer exercise belt for retaining said heating element;
wherein said neoprene lining is from about 2 mm to about 6 mm in thickness;
wherein said heating element is comprised of a flexible and continuous metal grid extending along a length of said compression band from a first end to an opposing second end;
wherein said heating element maintains an elevated temperature for generating and maintaining a heat application to said target body area for increasing a temperature of said target body area;
wherein said target body area includes an abdominal area and a hip of the user; and
a pocket sewn on said exterior surface for accommodating a rechargeable battery, wherein said rechargeable battery is removably stored in said pocket and provides electric power to said heating element.

9. The fat trimmer exercise belt of claim 8, wherein said first end comprises a loop fastener disposed on said interior surface of said compression band, and said second end includes a hook fastener on said exterior surface of said compression band for attachment of said compression band around the abdominal area and the hip of the user.

10. The fat trimmer exercise belt of claim 8, wherein said elevated temperature is from about 107° F. to about 135° F.

11. The fat trimmer exercise belt of claim 8, wherein said battery is a 450 mA battery.

12. The fat trimmer exercise belt of claim 8 further comprising an activation button disposed on said exterior surface for actuating said heating element, wherein an electric power is transferred from said battery to said heating element when said activation button is pressed for enabling said heating element to generate a heat to said target body area.

13. The fat trimmer exercise belt of claim 8, wherein said compression band has an elastic failure in the range of about 400% to about 1200% relative to a static length of said compression band.

14. A fat trimmer exercise belt for administering a thermal therapy and a soft tissue support to a target body area, the fat trimmer exercise belt comprising:
a compression band; and
a heating element for wearing around the target body area of a user;
wherein said compression band comprises a neoprene inner lining having a cavity disposed between an exterior surface and an interior surface of said fat trimmer exercise belt for retaining said heating element;
wherein said heating element comprises a first heating element portion around a center of the compression band, a second heating element portion proximal to a first end of said compression band, and a third heating element portion proximal to an opposing second end of said compression band;
wherein said first heating element portion is smaller than each of said second heating element portion and said third heating element portion;
wherein said second heating element portion and said third heating element portion provide more heat than said first heating element portion;
wherein said first heating element portion maintains an elevated temperature for generating and maintaining heat application to a first target body area;
wherein said second heating element portion maintains an elevated temperature for generating and maintaining heat application to a second target body area; and
further wherein said third heating element portion maintains an elevated temperature for generating and maintaining heat application to a third target body area.

15. The fat trimmer exercise belt of claim 14, wherein said second target body area is a first hip area of the user and said third target body area is a second hip area of the user.

16. The fat trimmer exercise belt of claim 15, wherein said first target body area is an abdominal area of the user.

17. The fat trimmer exercise belt of claim 16 further comprising a pocket sewn on said exterior surface for accommodating a rechargeable battery, wherein said rechargeable battery is removably stored in said pocket and provides an electric power to said first heating element portion, said second heating element portion and said third heating element portion.

18. The fat trimmer exercise belt of claim 17 further comprising an activation button disposed on said exterior surface for actuating said first heating element portion, said second heating element portion and said third heating element portion.

19. The fat trimmer exercise belt of claim 18, wherein said neoprene lining is from about 2 mm to about 6 mm in thickness.

20. The fat trimmer exercise belt of claim 19, wherein said compression band has an elastic failure in the range of about 400% to about 1200% relative to a static length of said compression band.
